(19) United States
(12) Patent Application Publication
(10) Pub. No.: US 2014/0221795 A1
(43) Pub. Date: Aug. 7, 2014

(54) MOBILE PLATFORM FOR OXIMETRY TESTING

(71) Applicant: Amber Yeager, Kansas City, MO (US)

(72) Inventor: Amber Yeager, Kansas City, MO (US)

(73) Assignee: BREATHE, LLC, Kansas City, MO (US)

(21) Appl. No.: 14/148,675

(22) Filed: Jan. 6, 2014

Related U.S. Application Data
(60) Provisional application No. 61/748,813, filed on Jan. 4, 2013.

(51) Int. Cl. G06F 19/00; A61B 5/00; A61B 5/1455

(52) U.S. Cl. CPC G06F 19/322; A61B 5/14551; A61B 5/0004
USPC 600/323; 705/3

(57) ABSTRACT

A computer assimilates and reports physiological data collected from a patient. The method includes the steps of recording physiological data collected from a patient's body via sensors with the sensors connected to diagnostic medical equipment. The diagnostic medical equipment transmits the physiological data to a computerized memory in a mobile computer device proximate the patient. Along with the physiological data, the mobile computer device receives test condition data into the computerized memory within the mobile computer device. The mobile computer device further displays a personal signature application to allow the patient to digitally sign electronic paperwork associated with the physiological data. The physiological data, the test condition data, and the electronic paperwork are then transmitted to a remote computer.

### BACKGROUND OF THE INVENTION

The invention relates to the field of medical equipment collecting a patient's physiological data at home or in an out-patient setting and requiring data transmission to a central location in a particular embodiment, the physiological data is related to oximetry testing.

Advances in medical monitoring equipment have led to numerous kinds of devices that patients use at home or on an out-patient basis to track physiological data. This data is gathered in real time via sensors attached to or in close proximity to the patient. The sensors transmit the data to a recording device, which often incorporates computerized memory, so that the data is available for further analysis by medical personnel. The data can be downloaded from the computerized memory to another computer or transmitted by conventional means over a network to a second computerized system (i.e., to a website that gives a medical professionals access to the original physiological data).

In one example that does not limit the scope of the invention disclosed herein, a patient may use medical equipment that measures the saturation of the patient's hemoglobin (i.e., via a pulse oximeter). This equipment is sufficiently non-invasive that it is often used at home with the data being collected without a medical professional being immediately present. The data is gathered for analysis by a DME provider who forwards the data to an independent diagnostic testing facility (IDTF) for processing. The IDTF personnel assimilate the data for a medical professional to use in treating the patient and for insurance companies to confirm that the treatment is covered by insurance policies. This process includes the step of processing an appropriate “assignment of benefits form” so that the IDTF can be paid for services by the health insurance carrier.

A step-by-step example shows how one prior art system operates:

1. The durable medical equipment provider (“the DME provider”) receives a prescription from a physician that orders an overnight oximetry test that a patient will conduct at home with the proper medical equipment.

2. By previous contractual arrangement, the DME provider has the proper credentials to create a patient profile. within a secure computer system owned and operated by an independent diagnostic testing facility (the IDTF) that will prepare official diagnostic reports from raw data collected from the medical equipment.

3. After the DME provider creates the patient profile, the IDTF computer system shows the patient as “bending” within the IDTF computer system.

4. The DME provider then faxes the prescription to the IDTF.

5. The IDTF verifies that the prescription is in proper order and either approves the prescription or marks it in error for follow up by the DME provider and the treating physician.

6. The DME provider utilizes the IDTF website and prints patient instructions and assignment of benefit forms which are necessary to complete the appropriate insurance payments. The IDTF computer system pre-fills the instructions and forms with the patient's demographic data.

7. The DME provider delivers a pulse oximeter machine to the patient along with the patient instructions and the assignment of benefit forms requiring signature.

8. The patient uses the medical equipment and measures oximetry overnight.

9. The next day, the DME provider picks up the pulse oximeter from the patient's home along with the manually signed assignment of benefits form.

10. The DME provider downloads the data from the pulse oximeter machine and transmits the raw data to the IDTF computer system for analysis.

11. The DME provider faxes the signed assignment of benefit form to the IDTF.

12. The IDTF posts the assignment of benefits form to the patient's file at the IDTF and faxes the oximetry report to the physician and the DME provider.

The prior art system brings forth a need in the industry for a more efficient data transfer from the oximetry equipment used by the patient such that the DME provider can avoid driving to the patient's home to retrieve equipment and further avoid connecting the retrieved equipment to a separate, remotely located computer for transmitting data to the IDTF. The use of manually signed assignment of benefit forms that are hand delivered or faxed is another step in the process in need of improvement.

### BRIEF SUMMARY OF THE INVENTION

In a first embodiment of a computerized method disclosed herein, a computer assimilates and reports physiological data collected from a patient. The method includes the steps of recording physiological data collected from a patient's body via sensors with the sensors connected to diagnostic medical equipment. The diagnostic medical equipment transmits the physiological data to a computerized memory in a mobile computer device proximate the patient. Along with the physiological data, the mobile computer device receives test condition data into the computerized memory within the mobile computer device. The mobile computer device further displays a personal signature application to allow the patient to digitally sign electronic paperwork associated with the physiological data. The physiological data, the test condition data, and the electronic paperwork are then transmitted to a remote computer.

In a second embodiment, a computer program product enables digital transmission of a patient's physiological data via a smart device application and includes computer controlling commands stored on a computer readable medium as an application for configuring a smart device to (i) receive physiological data from a patient's diagnostic medical equipment and (ii) receive test condition data from the patient; and (iii) transmit the physiological data and test condition data to a remote computer.

In a third embodiment, a computerized system processes physiological data and prepares an associated report; the system includes a diagnostic medical computer gathering physiological data from a patient. A mobile computer device proximate the patient has a computerized application for (i) receiving the physiological data from the diagnostic medical computer, (ii) receiving test condition data from the patient, and (iii) providing a digital signature function for completing electronic forms. The mobile computer device further includes a wireless data transmitter sending the data to a remote computer receiving the physiological data, the test condition data, and digital signature data associated with the patient.

### BRIEF DESCRIPTION OF THE DRAWINGS

FIG. 1 is a flow chart showing the steps of a computerized method of testing and transmitting oximetry data via hardware disclosed herein.

FIG. 2 is a front perspective view of an oximiter in the form of a finger probe used as diagnostic medical equipment as set forth herein.

FIG. 3A is a front perspective view of a control unit used to transmit oximetry data from the diagnostic medical equipment of FIG. 2.

FIG. 3B is a front plan view of a smart device with an application installed for retrieving and transmitting physiological data from the diagnostic medical equipment of FIG. 2.

FIG. 4 is a front plan view of patient record within the smart phone application of FIG. 2.

FIG. 5 is a front plan view of a sample of pending patient records accessible via the smart phone application of FIG. 2.

FIG. 6 is a front plan view of a set of control tabs within the smart phone application menu of FIG. 2.

FIG. 7 Is a screen shot of test condition data received by the IDTF from the smart phone application of FIG. 2.

FIG. 8 is a front plan view of a smart phone showing oximetry test results processed by the IDTF.

### DETAILED DESCRIPTION

For purposes of explaining the method and computerized system discussed below, terms are used in regard to the location of certain computers. A computer “proximate” a patient is distinguished from a “remote” computer a different distance from the patient. Terms such as “remote” and “proximate” are to be given their broadest interpretation without limiting the invention to any one embodiment or any particular geographical locations. Also, the term “smart” device is used to refer to a broad array of commercially available consumer products having sufficient computer hardware and software to be used for both signal processing and transmission of voice, text, image, internet, and numerous other kinds of data.

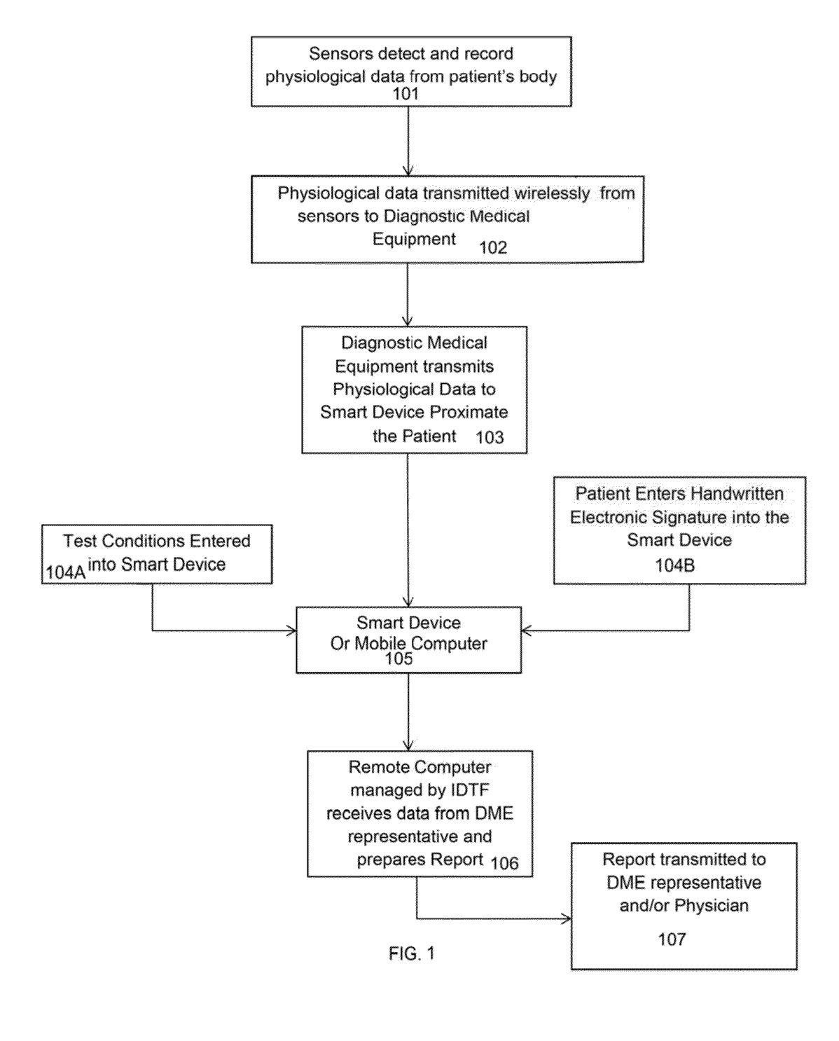

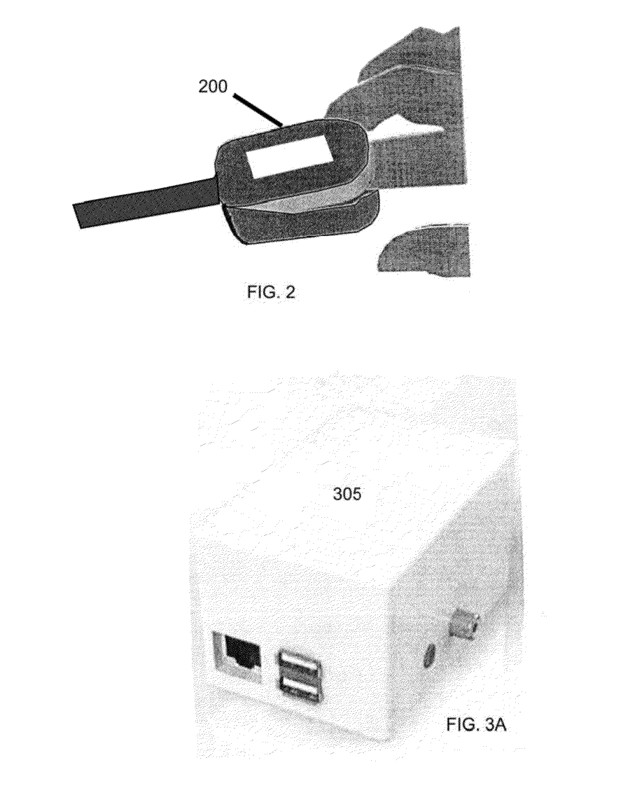

In a first embodiment, a computerized method as outlined in the Flow Chart of FIG. 1 is implemented via a smart, computerized device having appropriate hardware installed therein, including but not limited to processors and other non-transitory computer readable media. In one non-limiting example, a portable computer device (for example, a control unit (305) or a “smart” device (320)) is used in the system to retrieve data from diagnostic medical equipment (200) that is applied to or used in close proximity with a patient's body (e.g., an oximeter as shown in FIG. 2 including a sensor such as a probe connected to a patient). In one non-limiting example, the diagnostic medical equipment (200) communicates with either of the control unit (305) or the smart device (320), depending upon the set-up necessary for any given patient or any given diagnostic medical equipment representative. In all situations, the diagnostic medical device (200) communicates with either the control unit (305) or the smart device (320) is either wireless or wired communication to calculate oxygen saturation in a patient's bloodstream. The physiological data, such as the oximetry readings, is collected from the patient's body via sensors (e.g., pulse oximeter probes (200) attached to the patient's fingertip or elsewhere) that measure the patient's physiological response, such as blood oxygen level, in real time and transmit the data to an associated mobile computer device (305, 320) through either wireless e.g., (BLUETOOTH®) or conventionally wired data transmission.

In one example shown in FIG. 2, the oximeter (200) includes a probe that is attached to a region of the patient's skin, such as the fingertip or earlobe. The probe may include a light source, such as light emitting diodes, on one side of the probe and a light receiver, such as a photo-detector on an opposite side. The light sources shine light through the patient's tissues and the blood therein. The photo-detector on an opposite side of the tissue specimen responds to the different wavelengths of light transmitted through the tissue as indicative of the oxygen saturation of arterial blood flow with each pulse.

The diagnostic medical equipment (200) includes at least one processor within its computerized assembly and may include wireless data transmission capabilities, such as a BLUETOOTH® transmitter and/or receiver, such that the diagnostic medical equipment can transmit data from a sensor on the patient to another device within an appropriate wireless protocol. In one embodiment, the diagnostic medical equipment (200) sends the collected physiological data to a mobile computer device, such as either a control unit (305) or a smart device (320) used in the patient's presence or in close proximity to the patient.

Without limiting the invention disclosed herein, the mobile computer device (300) receiving the physiological data from the diagnostic medical equipment (200) is a mobile device with computer processing, data transmission, and telecommunications capabilities (i.e., a “smart device”). Smart devices include not only mobile laptop computers, but also smart phones, tablets, notebooks, iPads, personal digital assistants, and the like. A mobile computer device used to receive the physiological data from the diagnostic medical equipment can be any computerized device that has the capability to receive and transmit data via well-known telecommunications protocols, including cellular and “Wi-Fi” data transmission.

The mobile computer device may be a control unit (305) that has wireless technology transmission capabilities so that certain physiological data from the diagnostic medical equipment (200) can be transmitted to a remote computer outside the proximity of the patient wearing the sensor. In other words, the control unit (305) receives the raw physiological data, and the control unit (305) can push that data via Wi-Fi, cellular, or other transmission protocols to a remote computer. The remote computer is usually located at the site of an independent diagnostic testing facility (the IDTF) that will prepare official diagnostic reports from raw data collected from the medical equipment. In this way, a vendor or representative of a diagnostic medical device can easily transmit data to the IDTF after a testing period has been complete without driving the mobile computer device to a different location. In the case of blood oximetry, the control unit (305) can send blood oxygen saturation data directly to the IDTF from the tested patient's home where the test was performed.

The mobile computer device may also be a smart device (320) that a representative (the “DME” representative) for the company in charge of the diagnostic medical equipment carries to the location of the patient's test. Once the test is complete, the DME representative uses the smart phone (or any of the above noted mobile computer devices) to gather the physiological data collected by the sensors on the patient's body. Again, the data transmission from the diagnostic medical equipment to the mobile computer device may utilize a wireless protocol such as BLUETOOTH®.

Upon receiving the physiological data into the smart device (320) or the control unit (305), the DME representative may ensure that certain test condition data is also sent to the IDTF for processing. The test condition data (104) records the status of certain objective conditions related to the patient (i.e., location, medications in use, and the environmental conditions of the test). In one non-limiting example, the test condition data (104) is related to oximetry testing and includes notations of whether the test was performed overnight, for a particular duration, in an ambient air or room air environment, and whether oxygen was in use for the patient during the oximetry test. These test condition parameters (104) allow the IDTF to account for statistical features of the physiological data set so that the IDTF can apply appropriate filters, calibrations, scaling, and other data manipulation techniques to fully explore the meaning of the physiological data at hand.

In one embodiment of the computerized method of FIG. 1, the software utilized to implement the flow chart is installed onto a smart device (300) by conventional downloading operations and provides appropriate data entry screens (400, 500, 600) for a DME representative to enter the test conditions and other objective information about the patient under consideration and the testing environment in which the oximeter gathered data.

As another step in the automated testing process disclosed herein, the DME representative utilizing either the smart device (320) or the control unit (305) has the option of utilizing electronic paperwork stored in a computerized format and accessible by a personal computer or a smart device (320) at the patient's location. The digital paperwork may be resident on the personal computer or on the smart device or may he accessed from a remote server via the Internet or other data connection. In one embodiment, the electronic paperwork includes appropriate assignment of benefit forms that are necessary for insurance carriers to pay the proper entity for the patient's testing.

The mobile computer, or smart device, receiving the physiological data and test condition data also includes the appropriate functionality allowing the patient to sign the electronic paperwork, such as the assignment of benefits form, with a handwritten signature entered directly into the mobile computer device in digital format.

Upon receipt of the physiological data, the test condition data, and any required digital signatures from the patient, the mobile computer device (i.e., either the control unit (305) or the smart device (320) transmits these data sets to a remote computer (e.g., an IDTF computer) for processing. In the example of oximetry testing, the remote computer may be controlled by an independent diagnostic testing facility (an “IDTF) that processes all of the data transmissions received from the smart device or other mobile computer in use at the patient's location. The IDTF has the capability to process the raw data and generate a formal report related to the patient's physical condition.

The IDTF may receive additional data that identifies the mobile computer or smart device that is transmitting the patient's data. The additional data may be as simple as a telephone number for the DME representative's cell phone or it may be a more sophisticated identifier associated with a security protocol. In any event, the IDTF personnel matches the identifying data with the patient's record as a security measure to make certain that the physiological data, the test data, and the signature data received from the patient's location are paired with the appropriate patient record at the independent diagnostic testing facility.

The computerized method of gathering the physiological data, the test condition data, and the digital signature data may be implemented via a computer program product in the form of a smart device application (i.e., a smart phone “app”). The application allows the steps of the method described above to be implemented via a smart device at the patient's location in communication with a more robust computer or server at a remote IDTF location.

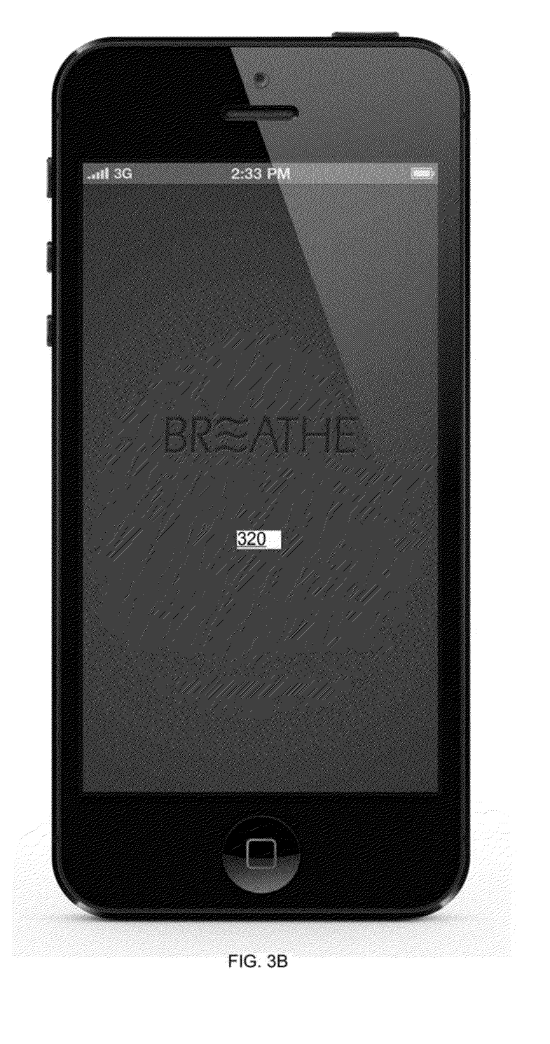

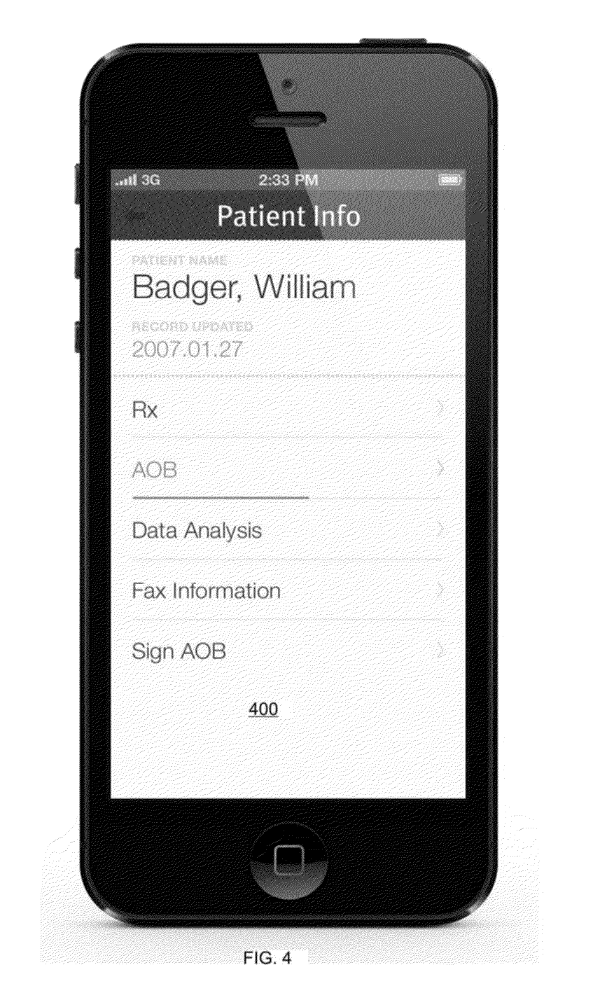

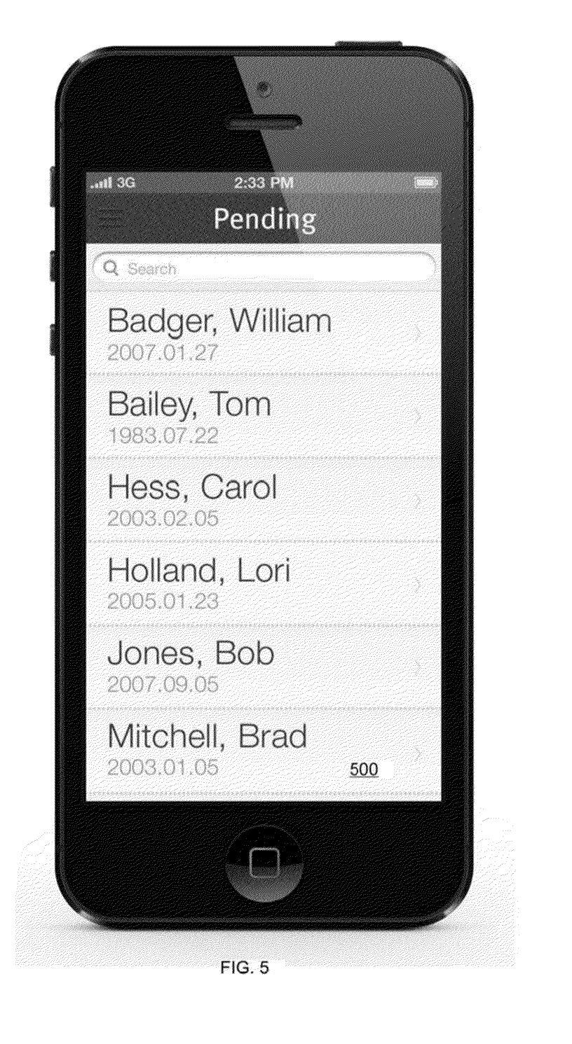

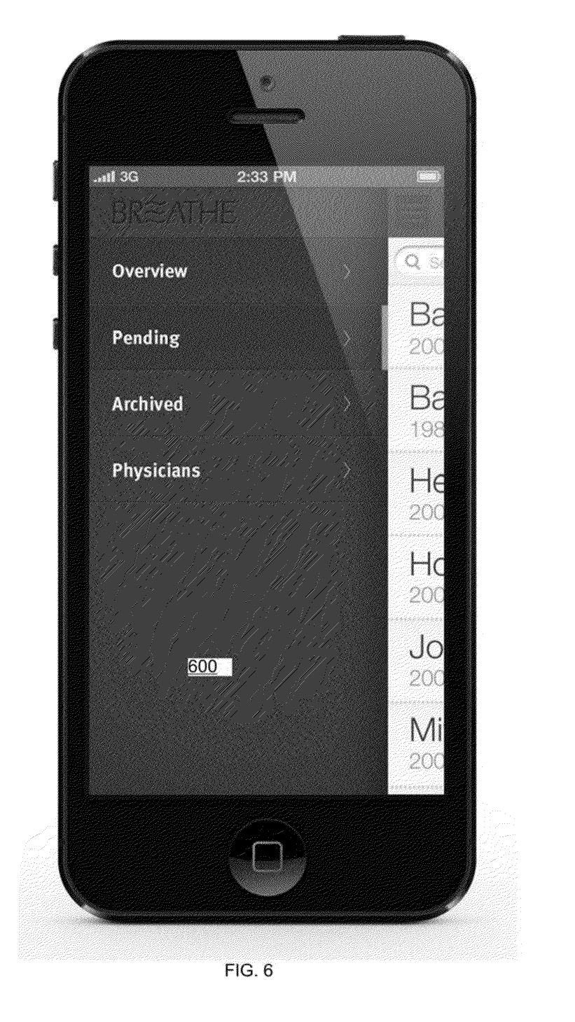

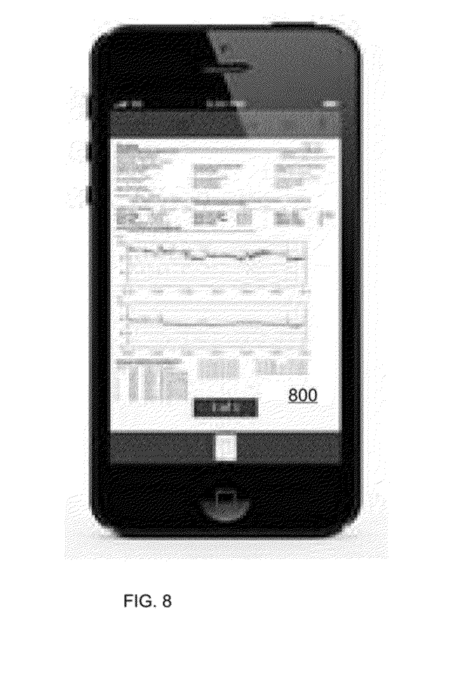

Without limiting the invention in any way, FIGS. 3-8 illustrate the above-described computer application implemented as a smart phone “app.” FIG. 3 is the screen on a smart phone that the DME representative would initiate at the patient's location to begin the data gathering process. In the example of FIG. 2, the data is oximetry data gathered at a patient location and ultimately sent to an IDTF. FIG. 4 shows that the smart phone application allows the DME representative to access patient information resident on a computer operated by the IDTF. Each patient that the DME representative is responsible for serving has a database entry stored on the remote computer operated by the IDTF. The remote IDTF computer communicates with the mobile device, or smart phone, operated by the DME representative, via standard wireless telecommunications protocols. FIGS. 5-8 show non-limiting examples of the kinds of screens available to the DME representative via the computerized application.

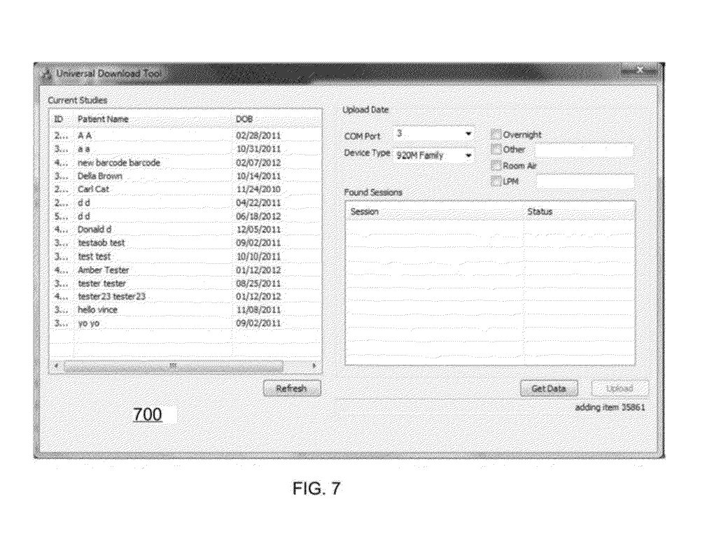

As noted above, the DME representative downloading data from the patient's test must ensure that test conditions are transmitted to the IDTF so that the processed results are accurate. FIG. 7 shows one example of the kinds of test conditions to be tracked, including the time of the test (i.e., “overnight”) and whether the patient's oximetry test was conducted while breathing ambient room air or liters per minute of prescribed oxygen. The IDTF processing test results has no direct access to the patient to gather or inter this test condition data, so recordation at the patient site is imperative to proper processing. In one embodiment, the diagnostic medical equipment senses the test conditions and transmits the test conditions with the physiological data to the mobile computer device in use by the DME rep. Otherwise, the smart device application may prompt either the patient or the DME representative to enter the test conditions for transmission to the IDTF's remotely operated computer.

In the end, the IDTF personnel receive the appropriate physiological data, test condition data, and any required handwritten signatures via electronic data transmission from the DME representative. The IDTF processes all of the data and generates a report after processing the raw data received from the patient's. location. FIG. 7 is an example of the kinds of reporting functions that the IDTF transmits back to the DME representative and/or the patient's physician.

The method, product, and system described herein are further explained in regard to the claims set forth below.

1. A computerized method of assimilating and reporting physiological data collected from a patient, the method comprising:
recording physiological data collected from a patient's body via sensors, said sensors connected to diagnostic medical equipment;
transmitting the physiological data from the diagnostic medical equipment to a computerized memory in a mobile computer device proximate the patient;
incorporating test condition data into the computerized memory within the mobile computer device;
displaying a personal signature application on the mobile computer device to allow the patient to digitally sign electronic paperwork associated with the physiological data;
transmitting the physiological data, the test condition data, and the electronic paperwork to a remote computer.

2. A computerized method according to claim 1, wherein the mobile computer device proximate the patient is a smart device.

3. A computerized method according to claim 1, wherein the mobile computer device proximate the patient is a smart device receiving data via Bluetooth transmission.

4. A computerized method according to claim 1, wherein said transmitting comprises wirelessly transmitting data from the mobile computer device to the remote computer.

5. A computerized method according to claim 1, further comprising processing the physiological data and the test condition data at the remote computer to generate a report.

6. A computerized method according to claim 5, further comprising transmitting the report to a physician or a diagnostic medical equipment representative or both.

7. A computer program product enabling digital transmission of a patient's physiological data via a smart device application, the computer program product comprising:
computer controlling commands stored on a computer readable medium as an application for configuring a smart device to (i) receive physiological data from a patient's diagnostic medical equipment and (ii) receive test condition data from the patient; and (iii) transmit the physiological data and test condition data to a remote computer.

8. A. computer program product according to claim 7, wherein said diagnostic medical equipment comprises a Bluetooth transmitter and said smart device comprises a Bluetooth receiver.

9. A computer Program product according to claim 7, wherein the smart telephone application comprises a digital signature function for signing electronic forms in digital data format.

10. A computer program product according to claim 8, wherein said smart telephone application transmits the physiological data, test condition data, and digital signature data to the remote computer.

11. A computerized system for processing physiological data and preparing an associated report, the system comprising:
a diagnostic medical computer gathering physiological data from a patient;
a mobile computer device proximate the patient and comprising a computerized application for (i) receiving the physiological data from the diagnostic medical computer, (ii) receiving test condition data from the patient, and (iii) providing a digital signature function for completing electronic forms, said mobile computer device further comprising a wireless data transmitter; and
a remote computer receiving. said physiological data said test condition data, and digital signature data associated with the patient.

12. A computerized system according to claim 11, wherein said diagnostic medical equipment gathers oximetry data.

13. A computerized system according to claim 11, wherein said mobile computer device comprises a smart device.

14. A computerized system according to claim 11, wherein said mobile computer device further transmits device identification data to said remote computer.

15. A computerized system according to claim 14, wherein said mobile computer is a smart phone and said identification data is a telephone number.

16. A computerized system according to claim 15, wherein said remote computer comprises a database of patient information and said remote computer verifies that transmitted data is from a recognized telephone number.

17. A computerized system according to claim 11, wherein said mobile computer device transmits said physiological data, test condition data, and digital signature data in a single data transmission.

18. A computerized system according to claim 11, wherein said test condition data comprises oxygen supplement data and test duration data.

19. A computerized system according to claim 18, wherein said test condition data is entered into the system in the presence of the patient.

20. A computerized system according to claim 19, wherein said digital signature function is enabled on said mobile computer device alter said test condition data is entered.
